(19) United States
(12) Patent Application Publication
(10) Pub. No.: US 2025/0321177 A1
(43) Pub. Date: Oct. 16, 2025

(54) SYSTEM FOR THE CARRYING OUT ANALYSES OF BLOOD SAMPLES WITH IMPROVED OPTOELECTRONIC SYSTEMS

(71) Applicant: DIESSE DIAGNOSTICA SENESE S.P.A., Milano (IT)

(72) Inventors: Sonia DE ANGELIS, Monteriggioni (SI) (IT); Daniela DIAMANTI, Monteriggioni (SI) (IT); Barbara FERRISI, Monteriggioni (SI) (IT); Mirko BERRETTI, Monteriggioni (SI) (IT)

(73) Assignee: DIESSE DIAGNOSTICA SENESE S.P.A., Milano (IT)

(21) Appl. No.: 18/868,928

(22) PCT Filed: Jun. 26, 2023
(86) PCT No.: PCT/EP2023/067251

§ 371 Date: Nov. 25, 2024

(30) Foreign Application Priority Data
Jun. 28, 2022 (IT) 102022000013630

(51) Int. Cl. G01N 15/05; G01N 15/01; G01N 33/49

(52) U.S. Cl. CPC G01N 15/05; G01N 15/01; G01N 33/49; G01N 2015/012

(57) ABSTRACT

A blood sample analysis system having a detection unit for optical absorption measurements on a blood sample in a tube, moving means to cause movement between the detection unit and the tube, and a processing unit. The processing unit commands the execution by the detection unit of two distinct optical absorption measurements on the blood sample. The processing unit sets parameters of the detection unit to create for each distinct measurement a reading curve corresponding to the absorption of radiation emitted by the detection unit as a function of the relative movement between the detection unit and the tube and to perform a comparison of the distinct reading curves based on one or more references and to select a single reading curve, and to estimate measurement parameters, such as for the measurement of erythrocyte sedimentation rate, starting from the selected single reading curve.

### FIELD OF APPLICATION

The present invention refers to a system for analyzing biological samples, in particular for measuring erythrocyte sedimentation rate in blood samples. The following description refers to this field of application with the only purpose of simplifying the exposition thereof.

### PRIOR ART

The measurement of the erythrocyte sedimentation rate (ESR) is a very common laboratory test for quickly identifying inflammatory processes. Specifically, the rate at which the erythrocytes in a blood sample settle at the bottom of a tube is evaluated.

As well known in this technical field, the reference calculation method of the ESR is the Westergreen method, which provides placing the blood to be analyzed diluted with sodium citrate in a graduated tube and measuring the sediment formed after one hour. Said calculation method therefore envisages using dedicated tubes and predetermined timings.

There are apparatuses which are able to measure the ESR also on standard tubes (for example the normal blood count tubes), in which, by optical absorption measurements on the blood sample in the tube, it is possible to obtain measurement values in line with the reference values of the aforementioned Westergreen method. In this case, the measurement is carried out in a relatively short time, for example the blood samples are allowed to stabilize for around minutes before the last reading is carried out.

In this type of apparatuses, it is very important to obtain absorption curves free of artifacts and irregularities (or at least to a really low level), as well as with such intensity and noise values as to guarantee an accurate analysis.

The technical problem of the present invention is to devise a system which has structural and functional features that are able to overcome the limits and drawbacks complained with regard to the prior art and which in particular is able to create reading curves which are always of high quality.

### SUMMARY OF THE INVENTION

The solution idea underlying the present invention is to develop a system suited for analyzing blood samples in tubes (also blood count standard tubes) by optical absorption measurements, wherein at least one optoelectronic unit is configured to acquire a plurality of absorption curves in relation to a determined sample (each curve being acquired in different conditions), while a processing unit is programmed for automatically selecting, among the various obtained absorption curves, a single curve having one or more desired determined features (for example particular features of shape, signal/noise ratio, etc.), so as to estimate the desired amounts (such as for example the ESR) starting from said optimal curve.

Based on said solution idea, the above-mentioned technical problem is solved by a system for the analysis of blood samples, comprising at least one detection unit configured to perform optical absorption measurements on a blood sample contained in a tube, moving means configured to cause a relative movement between the detection unit and the tube, and a processing unit configured to command the execution, by the detection unit, of at least two distinct optical absorption measurements on the blood sample, wherein, in each measurement, the processing unit is programmed to set respective parameters of the detection unit (for example radiation emission and/or detection parameters of the detection unit, also a single parameter), said parameters being different from measurement to measurement; to create, for each of said distinct measurements, a reading curve corresponding to the absorption of radiation emitted by the detection unit as a function of the relative movement between the detection unit and the tube; to perform a comparison of said distinct reading curves based on one or more references (therefore comparison among curves or also between the single curves and the references, without particular limitations); to select, based on said comparison, a single reading curve (said selected single reading curve being also indicated as optimal reading curve) among said distinct reading curves, and to estimate desired measurement parameters, such as for example parameters for the measurement of the erythrocyte sedimentation rate, starting from said selected optimal single reading curve.

More in particular, the invention comprises the following additional and optional features, taken individually or in combination if necessary.

According to an aspect of the present invention, the processing unit can be configured to discard reading curves that have not been selected.

According to an aspect of the present invention, the references can correspond to width values of the curve.

According to an aspect of the present invention, the references can correspond to shape factors (or form factors) of the reading curves, wherein said references can possibly be stored in a memory unit of the processing unit.

According to an aspect of the present invention, the processing unit can be configured to compare the obtained reading curves with reference curve models.

According to an aspect of the present invention, the processing unit can be configured to perform said comparison by executing an automatic learning procedure based on techniques of machine learning and/or artificial intelligence, for example based on neural networks.

According to an aspect of the present invention, the processing unit can be configured to acquire, for each blood sample, four reading curves.

According to an aspect of the present invention, the detection unit can comprise at least one emitter and a corresponding detector arranged so as to irradiate the tube and to collect the radiation after the same has passed through said tube, wherein said emitter is configured to emit radiation having an infrared or visible wavelength, or in general having any suitable wavelength.

According to an aspect of the present invention, in each distinct measurement, the processing unit can be configured to set respective different values of the radiation emitted by the emitter of the detection unit, in particular different intensity values.

According to an aspect of the present invention, the detection unit can be arranged on the moving means, which are configured to move said detection unit along the longitudinal axis of the tube so as to allow the acquisition of a plurality of measurement points along said longitudinal axis.

According to an aspect of the present invention, the system can comprise a support configured to support the tube, and an agitating element configured to agitate said tube. They can be separated components or they can be a single component.

According to an aspect of the present invention, the support of the tube can be comprised in a chain structure which is movable and defines a closed path for said tube, said chain structure comprising a plurality of supports for a corresponding plurality of tubes, which are integrally movable with said chain structure along an advancement direction.

According to an aspect of the present invention, the system can comprise four detection units arranged along the chain structure so that each of said four detection units is configured to analyze a tube moved by the chain structure at a corresponding time instant, wherein the processing unit can be configured to carry out the selection of the single reading curve for each of said four detection units.

According to an aspect of the present invention, the agitating element can comprise guides in engagement with portions of the chain structure, which is structured in portions that are connected to each other and are configured to rotate around an axis that is parallel to the advancement direction of the tubes, said agitating element comprising motorized means configured to move said guides and consequently to drag into rotation the portion of the chain structure engaged therewith.

According to an aspect of the present invention, based on the selected single reading curve, the processing unit can further be configured to define an ideal curve of the trapezoidal type adapted to approximate the selected reading curve, to carry out a procedure of optimization of said ideal curve, thereby generating an optimized ideal curve, and to generate, based on said procedure of optimization, parameters that are indicative of the erythrocyte sedimentation rate of the blood sample contained in the tube.

According to an aspect of the present invention, the processing unit can be configured to carry out the optimization of the ideal curve by a least-squares minimization according to the Levenberg-Marquardt algorithm.

The features and advantages of the system according to the invention will become apparent from the description, made hereinafter, of an embodiment example thereof given by way of an indicative and non-limiting example with reference to the attached drawings.

### BRIEF DESCRIPTION OF THE DRAWINGS

In said drawings:

FIG. 1 is a general scheme of a system for the analysis of blood samples according to the present invention;

FIG. 2 is a perspective view of an optical detection unit according to an embodiment of the present invention;

FIG. 3 is a top view of the system according to an embodiment of the present invention;

FIG. 4 is a perspective view of an agitating element for tubes according to an embodiment of the present invention;

FIG. 5 is a perspective view of a gripper of tubes and of a movement system thereof according to an embodiment of the present invention;

FIGS. 6A and 6B are examples of reading curves obtained with the system of the present invention; and

FIG. 7 is an example of a theoretic curve of the trapezoidal type adapted to approximate a real reading curve in an embodiment of the present invention.

### DETAILED DESCRIPTION

Referring to said figures, 1 globally and schematically indicates a system for the analysis of blood samples according to the present invention.

It should be noted that the figures represent schematic views and are not always drawn to scale, but are instead drawn so as to emphasize the important features of the invention. Further, in the figures, the various elements are represented in a schematic way and their shape can vary according to the desired application. It should also be noted that, in the figures, identical reference numbers refer to elements that are identical in shape or function. Finally, particular features described in relation to an embodiment illustrated in a figure can also be used for the other embodiments illustrated in the other figures.

It should also be noted that, unless explicitly indicated, the described process steps can also be reversed, if necessary.

The present invention provides a system for the analysis of blood samples, in particular for (but not limited to) the measurement of the erythrocyte sedimentation rate (ESR) in blood samples contained in a tube, identified with the reference P, which is not limited to a particular type. The tube P can indeed also be a normal blood count tube, but it should however be observed that the inventive aspects described herein are not limited to the aforementioned type of tube.

In its most general form, the present invention provides a system which is able to obtain reading curves by absorption of radiation, said curves being always of good quality so that the subsequent analysis step of said curves can be facilitated.

In order to enable carrying out the operations object of the present invention, the system 1 comprises a processing or control unit (identified with the reference C), which includes suitable memory units MEM and which is suitably programmed and designated for the management thereof, automatic control and analysis of measurement data. The processing unit C can be for example a computerized unit integrated in or external to the system and operatively connected thereto.

Furthermore, it should be noted that the processing unit C can be a single unit or can comprise a plurality of local and/or remote units, possibly communicating with each other, each one of them being designated for carrying out specific operations. The processing unit C is therefore able to control the system 1 for obtaining the desired analysis of the blood samples. Anyway, the present invention is in no way limited to the architecture used for the control unit C, which can generally be any suitable computerized unit, comprising one or more units according to the needs and/or circumstances.

It should also be noted that the term “system 1” refers to a generic analysis apparatus, provided with a suitable case and containing a plurality of components cooperating with each other in order to obtain the desired measurement and calculation operations, said apparatus not being anyway limited to a particular type.

Anyway, the present invention will be illustrated below with reference to a specific example in which the tubes P containing the samples to be analyzed are moved by a chain system along various reading stations, even if, as mentioned above, the teachings described herein are not limited to this embodiment and are also applicable to many other types of apparatuses having a different configuration.

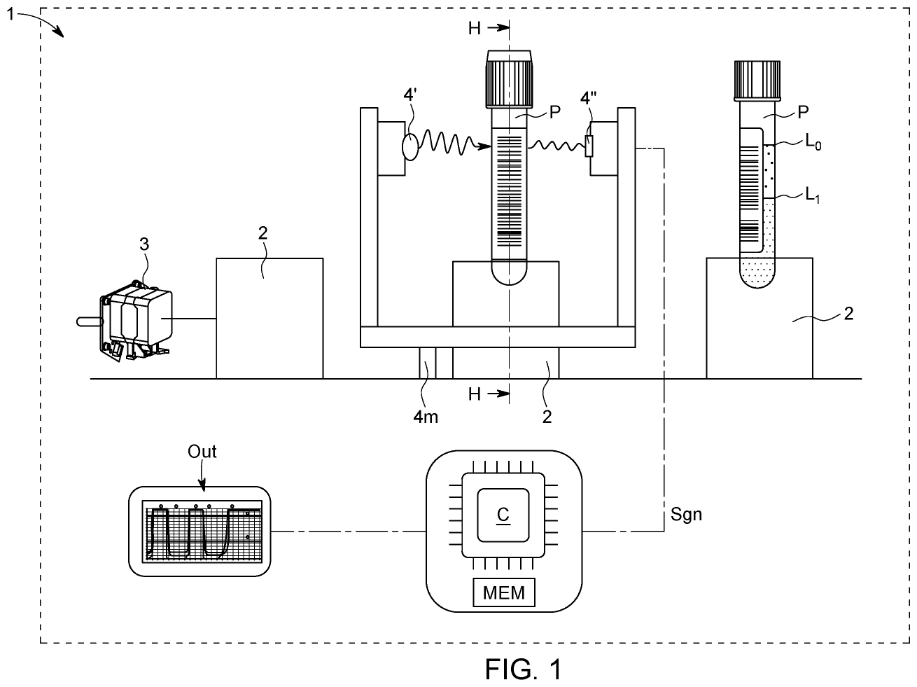

Referring to FIG. 1, the system 1, in its most general form, comprises a support 2 for housing at least one tube P containing a blood sample to be analyzed. In particular, the system 1 comprises a plurality of supports 2 for housing a corresponding plurality of tubes 2.

The system 1 furthermore comprises an agitating element 3 configured to agitate the tube P and to therefore allow the subsequent evaluation of the sedimentation process. The agitating element 3 is not limited to a particular configuration and substantially depends on the type of support used for housing and possibly moving the tubes. An example will be illustrated later on, in which the agitating element 3 cooperates with a movable chain structure on which the tubes P are arranged, without however limiting the scope of protection to said configuration; it should indeed be observed that, when the tubes are arranged on other types of supports, such as for example round-shaped plates, the agitating means are obviously different and adapted to the specific case.

There is then at least one optical detection unit (identified with the reference 4 and also called hereinafter reading unit or optoelectronic unit) configured to perform optical measurements, in particular optical absorption measurements, on the blood sample contained in the tube P.

In particular, the detection unit 4 comprises at least one emitter 4′ and a related detector 4″ arranged so as to irradiate the tube P with electromagnetic radiation and to collect the radiation after it has passed through said tube P. The emitter 4′ is preferably a LED configured to substantially emit white light, such that the presence of labels on the tube P (and other external factors) does not affect the measurement.

Obviously, the emitter 4′ is not limited to the type indicated above, for example, it can be any light source configured to emit radiation at any suitable wavelength, and the detector 4″ can therefore be accordingly selected. For example, the emitter 4′ can be a LED configured to emit radiation in the infrared or visible wavelength (or in general at any suitable wavelength), as well as it can also be a laser of any type and any suitable wavelength (and, therefore, it is possible to use a source with different coherence features).

The detection unit 4 therefore allows, through absorption measurements on the blood sample in the tube P, to obtain reading curves which will be used as starting point of the calculation procedure, as will be detailed hereinafter.

Suitable moving means 4m configured to cause a relative movement between the detection unit 4 and the tube P during the optical absorption measurement are further provided so as to irradiate said tube P in different portions, and therefore so as to obtain a plurality of n discrete measurement points (identified with the subscript i) forming the reading curves. In particular, the moving means 4m are configured to cause a step movement of the detection unit 4 (for example a lifting and lowering movement with respect to the longitudinal axis H-H of the tube P). As will be detailed hereinafter, the obtained reading curves are representative of the light intensity detected as a function of the reading steps (said steps being possibly convertible in time instants).

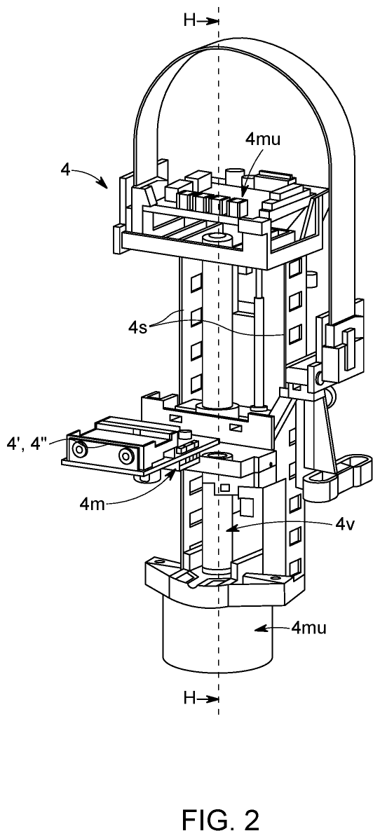

As illustrated in FIG. 2, which shows a non-limiting example of the reading unit 4, the moving means 4m can comprise a trolley moved by a suitable motor unit 4mu (comprising an own control driver board, which is also identified with the reference 4mu). The detection unit 4 (in particular both the emitter 4′ and the detector 4″) is therefore arranged on the trolley 4m, thus allowing a movement thereof in a direction which is substantially parallel to the longitudinal axis H-H of the tube P, and therefore allowing the acquisition of the plurality of measurement points along said longitudinal axis H-H. In this example, the motor unit 4mu moves an endless screw 4v, which in turn causes the movement of the trolley 4m. Further, in the example of FIG. 2, the emitter 4′ and the detector 4″ are both moved by the trolley 4m, said trolley being suitably shaped so as to allow the housing of the tube (not illustrated in FIG. 2) in a substantially central position thereof. All the aforementioned components are supported by a support 4s, which therefore acts as load-bearing structure of the detection unit 4.

It should however be noted that the present invention is in no way limited to the configuration of the detection unit 4, and therefore it is possible to adopt any other suitable configuration, for example in relation to the movement of the detectors/emitters or their arrangement.

In an embodiment of the present invention, when a plurality of reading units are present, it is possible to carry out the related calibration (alignment) between the various reading units by adjusting the positioning of the emitter/detector with respect to a testing tube (not illustrated in the figures), acting on adjustment means so as to adapt the reading curves, in particular the detected light intensity, with respect to the references provided by said testing tube.

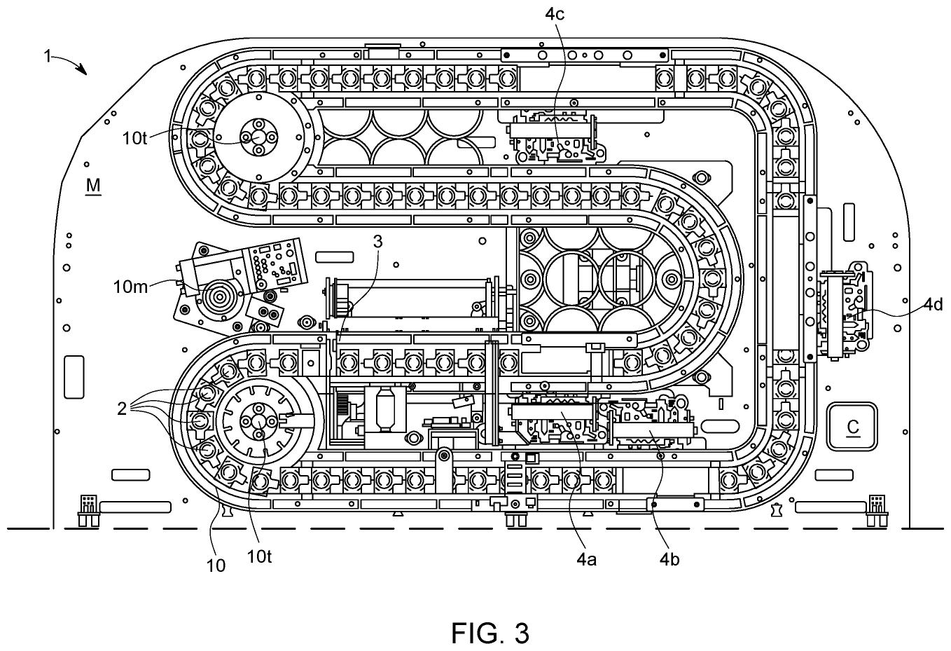

Referring now to FIG. 3, in a particular embodiment of the present invention, as previously mentioned, the system 1 comprises a chain structure 10 on which the supports 2 for the tubes P are formed. The chain structure 10 is movable and defines a closed path for the tubes P, said closed path substantially laying on a horizontal plane, for example parallel to the surface on which the system 1 is arranged.

The chain structure 10 comprises a plurality of portions connected to each other, each portion providing a support for a respective tube P. In an embodiment, the chain portions 10 are connected to each other by a ball joint and can rotate with respect to each other.

Thereby, the supports 2 for the tubes P are included in the chain structure 10 which is movable and defines the closed analysis path of said tubes P, said chain structure 10 comprising a plurality of supports for a corresponding plurality of tubes P, which are therefore integrally movable therewith.

In a particular embodiment, the system 1 comprises at least two detection units, preferably four detection units, arranged along the chain structure 10, such that each one of said four detection units is configured to analyze a tube P moved by the chain structure 10 in a specific time instant when the tube passes through at it (and the sensors are suitably lifted/lowered by the motor unit).

In particular, a first detection unit 4a acquires a first reading curve soon after agitating the tubes (therefore carrying out a reference reading) while a second reading unit 4b, arranged in a different position along the chain 10, carries out a measurement after the tube P has passed through for a specific sedimentation time, in particular after twenty minutes. Two further detection units can also optionally be present and arranged at intermediate points of the chain structure 10, so as to perform measurements also at intermediate time instants (for example at minutes twelve and seventeen).

To sum up, in the embodiment of FIG. 3, the analysis module M of the system 1 comprises the chain structure 10, which can have for example eighty-nine mashes in which the tubes P are inserted, said mashes being free to rotate in their junction point. The chain 10 rotates clockwise inside the analysis module by means of two traction wheels 10t moved by a motor unit 10m, transferring the tubes P to an agitating unit and subsequently to the optoelectronic units.

The movement speed of the chain structure 10 is set so as to allow the samples to stabilize for twenty minutes before the last reading is carried out. The reading units that are present in the analysis module are preferably four: the first reader is located immediately after the agitating element, the second reader is located such that the samples are read after twelve minutes, the third reader is located in the position which corresponds to an analysis time equal to seventeen minutes, and the fourth reader is located near the output position of the sample at a reading time equal to twenty minutes.

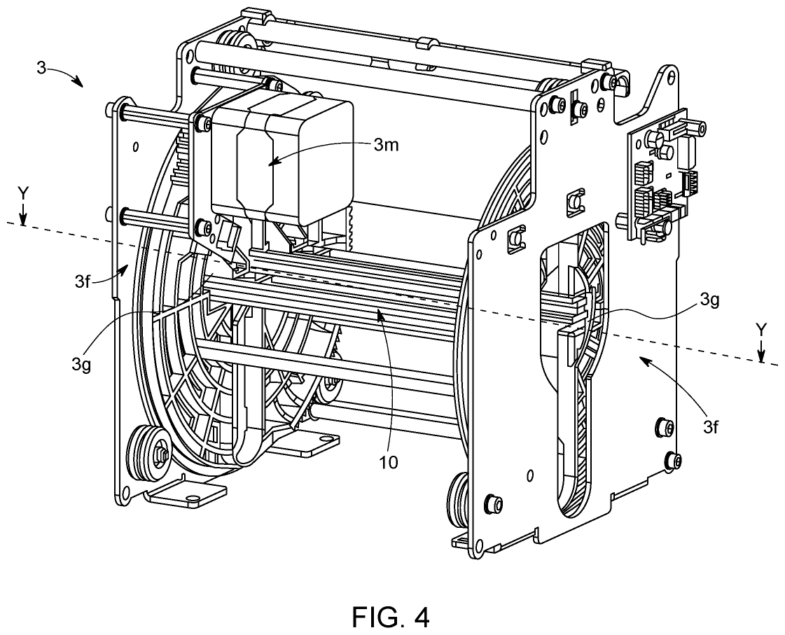

As mentioned, before carrying out the reading of the blood samples, the tubes are agitated by the agitating or mixing element, indicated herein with the reference 3. As illustrated in FIG. 4, the agitating element 3 can comprise guides 3g in engagement with parts (for example side tracks) of the chain structure 10, which—as seen above—is structured in various portions which are connected to each other and are configured to rotate around an axis Y-Y that is parallel to the direction of advancement of the tubes P. Suitable motorized means 3m are furthermore present, which means are configured to move said guides 3g through a suitable gear system and consequently to drag into rotation the portion of the chain structure 10 engaged therewith. Essentially, a guide-pads system is therefore created which allows agitating the tubes P. As illustrated in the non-limiting example of FIG. 4, the guides 3g are formed as two flanges 3f, for example round-shaped, which integrally move by means of the motorized means 3m.

As mentioned several times, the present invention is in no way limited to the structure of the components of the system 1 and many other embodiments are possible. For example, in an embodiment which is not illustrated in the figures, the support 2 and the agitating element 3 can also be a single component, adapted both to support and to agitate the tubes P, of any suitable shape.

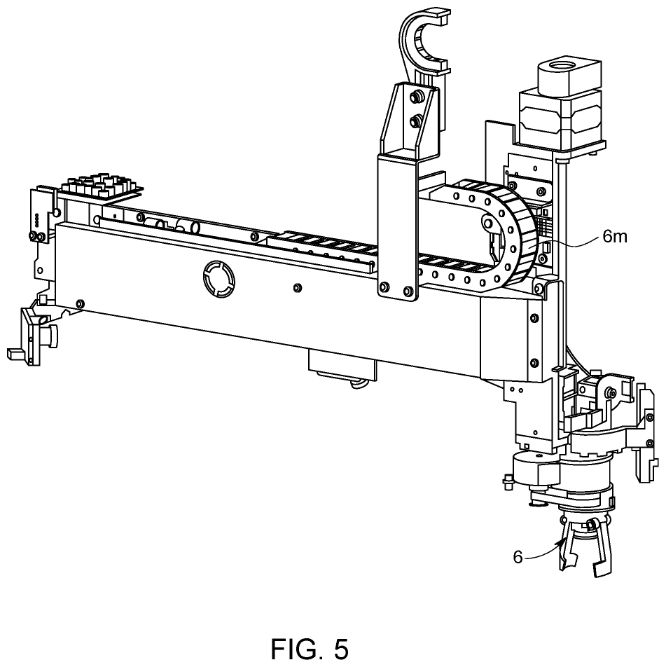

Referring now to FIG. 5, in some embodiments, the system 1 can also comprise a gripper 6 which is moved by suitable means 6m (controlled by suitable control means) and is configured to pick up the tubes P from a respective rack (not illustrated in the figures) and to arrange them in the support 2 on the chain structure 10.

A housing area for racks containing the various tubes P to be analyzed can also be present, as well as control means can also be present which are configured to verify the correct positioning of the various racks in said housing area. There is also a photo camera which acquires images of the racks and, after the processing of said images, the processing unit is able to verify the presence of the tubes in the rack and the position thereof, so as to be able to communicate this information to the control means of the gripper 6.

Anyway, as mentioned above, whatever the support and movement mode of the tubes P may be, the present invention envisages processing, through the processing unit C, the signals (identified with the reference Sgn) from the detection unit 4 which contain the information on the radiation absorption by the blood sample for creating and subsequently processing the reading curves.

In particular, based on said signals Sgn, the processing unit C is configured to firstly create the reading curve (identified with the reference L) corresponding to the real absorption of the radiation emitted by the detection unit 4. As mentioned above, the reading curve L comprises a plurality of n measurement points, acquired as a function of the relative movement between the detection unit 4 and the tube P, for example by the step movement of the motorized trolley 4m which supports the detection unit 4 as in FIG. 2. In other words, the experimentally obtained reading curve L is made up of n points (that is Li, with i=0, . . . , n−1).

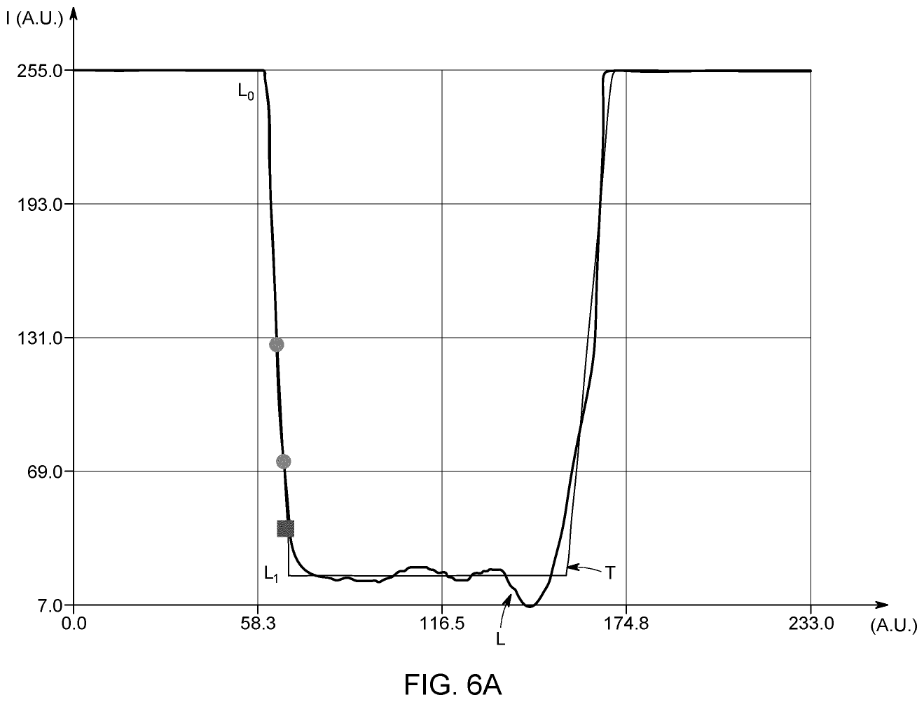

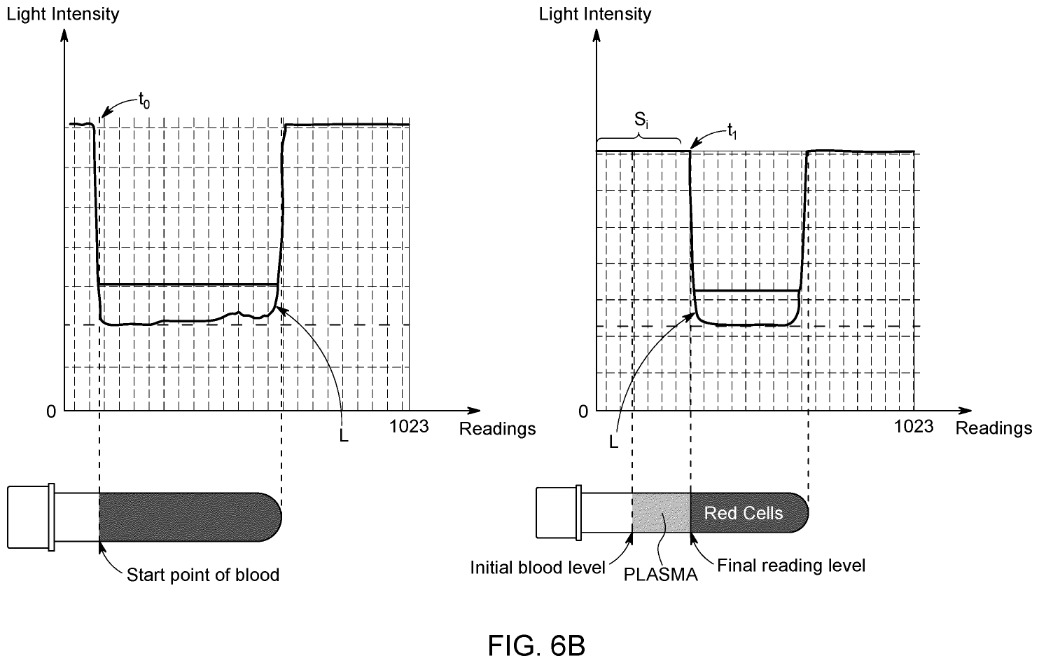

As mentioned above, the reading curves L are acquired immediately after agitating the tubes (reference) and then after a specific sedimentation time (up to twenty minutes), in order to provide information on the sedimentation process of the blood sample in the tube P. In general, the reading curve L shows, in the transition from plasma to sediment, a clear variation of the intensity of the measured radiation, in particular the absorption of radiation is higher at the sediment, thereby causing a decrease in the detected light intensity. There is then the presence of a plateau up to the bottom of the tube, as illustrated in the example of FIGS. 6A and 6B, which show examples of reading curves acquired through a detection unit 4 according to an embodiment of the present invention.

Even more in particular, in the reading subsequent to the reference one, that is the one occurring after a specific sedimentation time, it can be noted that the decrease in intensity due to the absorption of radiation occurs in a point ti subsequent to the point to in which the first reference reading occurs (that is at subsequent motor steps, and therefore in a subsequent time instant), due to the sediment which forms after a certain period of time (as illustrated in the example of FIG. 6B, in which it can be seen that the beginning of the plateau due to the sedimentation and the consequent absorption of the blood cells is displaced more to the right: as mentioned, this is due to the sedimentation, that is the separation from the rest of the plasma, in which there is no significant absorption of radiation, as can be seen in the first part Si of the right graph of FIG. 6B), therefore providing necessary information for estimating the erythrocyte sedimentation rate. The control unit C therefore comprises processing means (for example suitable software modules) for processing the desired measurements according to predefined protocols, thereby allowing the kinetic analysis of the blood samples. In particular, the calculation of the ESR is associated with the decreasing phase of the curves, which is the reason why it is very important to have optimal curves in which said trends are clear, which is obtained by means of the present invention, as illustrated below.

Advantageously according to the present invention, the processing unit C is configured to command the execution, by the reading unit 4, of at least two distinct optical absorption measurements on the blood sample. In a particular embodiment of the present invention, the processing unit C is configured to acquire, for each single detection unit 4 and for each blood sample, four reading curves.

More in particular, in each measurement, the processing unit C is programmed to set specific emission and/or detection parameters of the detection unit 4, said parameters varying from measurement to measurement. In an embodiment, in each distinct measurement, a determined intensity value of the radiation emitted by the emitter 4 is set, for example increasing from measurement to measurement. In other words, each detection unit 4 carries out a determined number of readings (preferably four) of each sample, for example up-down-up-down readings, wherein different values of the radiation emitted by the emitters 4′ of the optoelectronic units 4 can be set, so as to have for each unit a set of different absorption curves.

In a preferred embodiment, as mentioned above, the intensity of the source is varied, but the possibility of varying also other parameters of said source or also of the detector is not excluded (for example, the wavelength of the source, the gain of the detectors, etc.).

For each of said distinct measurements, the calculation module of the processing unit C is therefore configured to create a corresponding reading curve L indicating the absorption of radiation by the blood sample.

Suitably, the processing unit C is then configured to perform a comparison of said distinct reading curves based on one or more references. It can immediately be noted that the term “reference” is to be understood in a broad sense and can comprise a comparison between the same measurement curves, in which particular aspects of said curves are evaluated, or a comparison with reference curves, without being limited to a particular comparison and analysis procedure.

For example, the reference values can comprise the width of the curve, in which case the reading curve having the maximum width, that is the curve for which there is a higher detected intensity with respect to the noise background of the measurement, is selected.

Another reference parameter, strictly connected to the previous one, is the signal-noise ratio, being understood as the ratio between the average intensity of signal and the background noise, measured, for example, as the RMS (acronym for “root mean square”) for the background signal of the detector 4″.

In addition or alternatively, other references which can be used for the comparison can be the shape factors of the reading curves, for example the presence of excessive irregularities, undesired oscillations, saturation phenomena and so on. A parameter which can be evaluated is the presence or absence of spikes, which can be evaluated for example based on the presence of excessive variations between adjacent measurement points, that is variations higher than a certain threshold.

All the above-mentioned references (such as thresholds, reference intensity values and similar) can be stored in the memory unit MEM of the processing unit C.

Further, in an embodiment of the present invention, the processing unit C is configured to compare the reading curves obtained with models of reference curves, thereby selecting the curve closer to the reference model. In this case, the processing unit C can be programmed to perform the aforementioned comparison by executing an automatic learning procedure based on techniques of machine learning and/or artificial intelligence, for example based on neural networks. In other words, in this embodiment, artificial intelligence algorithms are implemented, such as for example the neural networks which, if trained with a suitable dataset of curves, automatically allow to discriminate the best curve.

It can furthermore be noted that the above-mentioned operation of acquisition of multiple curves and comparison curves is valid for each detection unit 4 of the system 1, regardless of their number.

Based on said comparison, a single optimal reading curve (identified as Lₒₚₜ) is then selected, that is the curve which is considered to be the best, while the other reading curves are discarded.

Thereby, it is possible to estimate desired measurement parameters, such as for example the parameters for the measurement of the erythrocyte sedimentation rate, starting from said selected single reading curve Lₒₚₜ, suitably processing said curve.

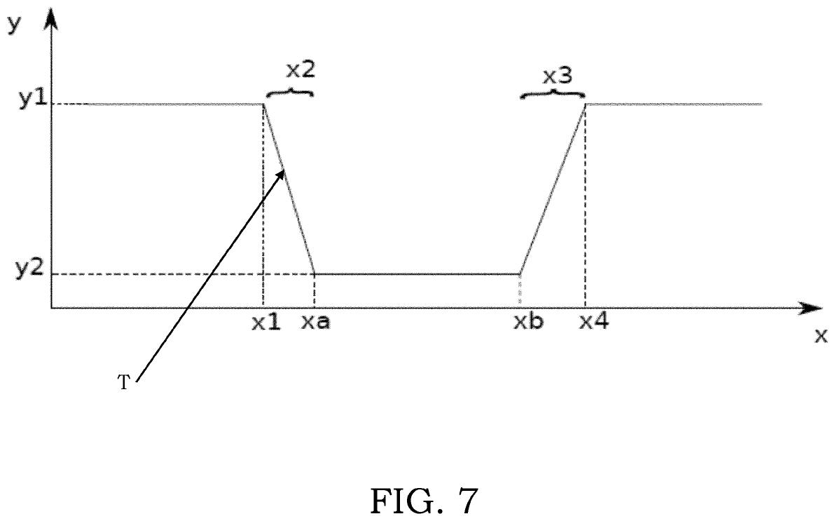

For example, in an embodiment, in order to measure the erythrocyte sedimentation rate, the processing unit C is configured to define an ideal curve (identified with the reference T, as schematically shown in FIG. 7) of the trapezoidal type adapted to approximate the optimal reading curve Lₒₚₜ. An optimization procedure of this ideal curve T is then performed, thereby generating an optimized ideal curve and, based on this optimization procedure, it is possible to obtain all the desired parameters, for example the parameters that are indicative of the erythrocyte sedimentation rate. In a particular example, the processing unit C can be configured to carry out the optimization of the ideal curve T by a least-squares minimization according to the Levenberg-Marquardt algorithm.

To conclude, the present invention therefore allows to brilliantly overcome the technical problem, providing the system and method as above and solving all the drawbacks of the prior art.

Advantageously, a simple yet highly effective auto-calibration procedure of the optoelectronic units, which are present inside the analysis module of the system, is provided, thereby always obtaining curves of optimal quality, and therefore considerably improving the analysis process, since said process is always based on optimal curves that are automatically selected by the system.

Suitably, the best curve is automatically discriminated for each sample, said curve being used for the subsequent measurements; this is obtained by determining suitable features of the curves, such as for example by determining the width of the curve or the absence of spikes, or by techniques of machine learning.

Obviously, a person skilled in the art, in order to satisfy contingent and specific needs, can make various modifications and variations to the system described above, which modifications and variations are all included in the scope of protection of the invention as defined by the following claims.

1. A system for the analysis of blood samples, comprising:
a detection unit configured to perform optical absorption measurements on a blood sample contained in a tube;
moving means configured to cause a relative movement between the detection unit and the tube; and
a processing unit configured to:
command the execution, by the detection unit, of at least two distinct optical absorption measurements on the blood sample, wherein, in each measurement, the processing unit is programmed to set respective parameters of the detection unit, said parameters being different from measurement to measurement;
create, for each of said distinct measurements, a reading curve corresponding to the absorption of radiation emitted by the detection unit as a function of the relative movement between said detection unit and the tube;
perform a comparison of said reading curves obtained from said distinct measurements based on one or more references;
based on said comparison, select a single reading curve among said reading curves from said distinct measurements; and
estimate desired measurement parameters starting from said selected single reading curve.

2. The system according to claim 1, wherein the processing unit is configured to discard reading curves that have not been selected.

3. The system according to claim 1, wherein the references correspond to width values of the curve.

4. The system according to claim 1, wherein the references correspond to shape factors of the reading curves, said references being stored in a memory unit (MEM) of the processing unit.

5. The system according to claim 1, wherein the processing unit is configured to compare the obtained reading curves with reference curve models.

6. The system according to claim 5, wherein the processing unit is configured to perform said comparison by executing an automatic learning procedure based on techniques of machine learning and/or artificial intelligence.

7. The system according to claim 1, wherein the processing unit is configured to acquire, for each blood sample, four reading curves.

8. The system according to claim 1, wherein the detection unit comprises an emitter and a corresponding detector arranged to irradiate the tube and to collect the radiation after the same has passed through said tube, wherein said emitter is configured to emit radiation having an infrared or visible wavelength.

9. The system according to claim 8, wherein, in each distinct measurement, the processing unit is configured to set respective different values of intensity of the radiation emitted by the emitter of the detection unit.

10. The system according to claim 1, wherein the detection unit is arranged on the moving means, which are configured to move said detection unit along the longitudinal axis of the tube, and to thereby allow the acquisition of a plurality of measurement points along said longitudinal axis.

11. The system according to claim 1, comprising a support configured to support the tube, and an agitating element configured to agitate said tube.

12. The system according to claim 11, wherein the support of the tube is comprised in a chain structure which is movable and defines a closed path for said tube, said chain structure comprising a plurality of supports for a corresponding plurality of tubes, said tubes being integrally movable with said chain structure along an advancement direction.

13. The system according to claim 12, comprising four detection units arranged along the chain structure so that each of said four detection units is configured to analyze a tube moved by the chain structure at a corresponding time instant, wherein the processing unit is configured to carry out the selection of the single reading curve for each of said four detection units.

14. The system according to claim 12, wherein the agitating element comprises guides in engagement with portions of the chain structure, which is structured according to portions that are connected to each other and are configured to rotate around an axis that is parallel to the advancement direction of the tubes (P), said agitating element comprising motorized means configured to move said guides and consequently to bring into rotation the portion of the chain structure engaged therewith.

15. The system according to claim 1, wherein, based on the selected single reading curve, the processing unit is further configured to:
define an ideal curve of the trapezoidal type adapted to approximate the selected single reading curve;
carry out a procedure of optimization of said ideal curve, thereby generating an optimized ideal curve (T′); and
generate, based on said procedure of optimization, parameters indicative of the erythrocyte sedimentation rate of the blood sample contained in the tube,
wherein the processing unit is configured to carry out the optimization of the ideal curve by a least-squares minimization according to the Levenberg-Marquardt algorithm.
